(19) United States
(12) Patent Application Publication
(10) Pub. No.: US 2009/0182347 A1
(43) Pub. Date: Jul. 16, 2009

(54) IMPLANTATION METHODS, SYSTEMS AND TOOLS FOR CARDIAC LEADS ASSOCIATED WITH INTRAVASCULAR IMPLANTABLE DEVICES

(76) Inventors: Terrance Ransbury, Chapel Hill, NC (US); Kevin Holbrook, Chapel Hill, NC (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 12/327,791

(22) Filed: Dec. 3, 2008

Related U.S. Application Data
(60) Provisional application No. 61/005,354, filed on Dec. 3, 2007.

(51) Int. Cl. A61B 17/00

(52) U.S. Cl. 606/129

(57) ABSTRACT

Improved methods, systems and tools for implanting cardiac leads associated with intravascular implantable devices (IID) within a patient's heart are disclosed. A method is described for implanting a cardiac lead within a patient's heart wherein the proximal end of the cardiac lead is not accessible for introduction of a steerable stylet. A delivery device is described for guiding a cardiac lead to a desired implant location, the delivery device including a grasper mechanism for releasably holding the cardiac lead. The grasper mechanism is on the distal end of an elongated flexible body, and is operated with a handle located on the proximal end of the flexible body.

### RELATED APPLICATIONS

The present invention claims priority to U.S. Provisional Patent Application No. 61/005,354, entitled “Implantation Methods, Systems and Tools for Intravascular Implantable Devices,” filed Dec. 3, 2007 which is hereby incorporated by reference in its entirety.

### FIELD OF THE INVENTION

The present invention generally relates to devices, systems and methods for diagnosing and treating patients. In particular, the invention relates to methods, systems and tools for implanting cardiac leads associated with long-term active therapeutic medical devices implanted in the vasculature of a patient.

### BACKGROUND OF THE INVENTION

Implantable devices that provide long-term active therapies such as artificial pacemakers, defibrillators, and implantable cardioverter defibrillators (“ICDs”) have been successfully implanted in patients for years for treatment of heart rhythm conditions. Pacemakers are implanted to detect periods of bradycardia and deliver low energy electrical stimuli to increase the heart rate. ICDs are implanted in patients to cardiovert or defibrillate the heart by delivering high energy electrical stimuli to slow or reset the heart rate in the event a ventricular tachycardia (VT) or ventricular fibrillation (VF) is detected. Another type of implantable device detects an atrial fibrillation (AF) episode and delivers an electrical stimuli to the atria to restore electrical coordination between the upper and lower chambers of the heart. The current generation for all of these implantable cardiac rhythm management (CRM) devices are typically can-shaped devices implanted under the skin that deliver electrical stimuli via leads that implanted in the heart via the patient's vascular system.

Conventional pacemakers and ICDs are implanted subcutaneously, typically in the pectoral region. Conventional implantable pulse generators such as pacemakers and ICDs use conventional leads in the form of elongated, floppy lead bodies that insulate, seal and protect one or more conductors configured to transmit electrical pulses between the pulse generator and one or more electrodes on the lead.

To implant the one or more intravascular leads for a conventional device implanted subcutaneously in the pectoral region, the lead is passed into the subclavian vein, routed through the superior vena cava, and down into the heart. Most intravascular cardiac leads for conventional CRM devices are guided with use of a stylet that is inserted into a lumen within the lead body accessed via the proximal end of the lead, with the stylet used to direct the distal end of the lead into the desired position. The lead tip is affixed in, on, or near the heart, depending on the desired treatment.

Once in position, the distal end of the lead may be fixed in position within the heart, either by passive fixation or active fixation. Passive fixation leads may feature protruding tines, and/or a steroid-coated lead tip, such that when the lead tip is inserted to the desired location, biological processes in the heart tissue will secure the lead in place. Active fixation leads typically include a helix or corkscrew tip, and this tip is secured directly into the myocardium. Active fixation offers more precise placement of the lead, as well as greater stability when secured in the heart.

The one or more leads associated with a conventional CRM device are typically not integrated with the device; instead, a header is provided on the device for connecting the one or more leads to the device. Such a connection arrangement between the conventional device and the lead allows for access to the lumen within the lead via the proximal end. Implantation of the device typically follows implantation of the lead. The lead is connected to the device, and the device is then secured in the patient.

While stylet-based delivery of cardiac leads is the most prevalent technique used, other techniques for cardiac lead delivery and fixation have also been developed. One such technique is an over-the-wire technique in which the lead is advanced over a guide wire. Different versions of this over-the-wire technique are described, for example, in U.S. Pat. Nos. 5,003,990, 5,304,218 and 6,129,749. Another technique involves the use of a guide catheter as a pusher for delivering the lead into position within the heart. Different versions of a guide catheter technique are described in U.S. Pat. Nos. 5,571,161, 6,185,464, 7,018,384 and 7,092,765.

Next generation long-term active implantable devices may take the form of elongated intravascular devices that are implanted within the vascular system of a patient, instead of under the skin. Examples of these intravascular implantable devices (IIDs) are described, for example, in U.S. Pat. No. 7,082,336 and U.S. Published Patent Application Nos. 2005/0043765A1, 2005/0228471A1 and 2006/0217779A1. These devices contain electric circuitry and/or electronic components that must be hermetically sealed to prevent damage to the electronic components and the release of contaminants into the bloodstream. Due to the length of these implantable devices, which in some cases can be approximately 10-60 cm in length, the devices must be flexible enough to move through the vasculature while being sufficiently rigid to protect the internal components.

Due to the many differences between conventional implantable CRM devices and intravascular implantable devices, well-known methods and devices for traditional lead introduction and fixation are not necessarily applicable to next-generation IIDs. For example, lead(s) for conventional CRM devices are usually introduced into the heart by way of the superior vena cava, while for intravascular implantable devices, the lead(s) are usually introduced to the heart via the inferior vena cava. The maneuvering of the lead from the inferior vena cava into the right atrium and on into the right ventricle is especially problematic using prior lead delivery systems and methods. Additionally, the cardiac lead of an IID is unlike a cardiac lead for a conventional CRM device in that the proximal end of the lead is generally unavailable for access to aid in the implantation of the lead, such as by use of a steerable stylet introduced into a lumen of the lead through the proximal end of the lead.

Previous approaches for delivering cardiac leads into the heart for intravascular implantable devices are disclosed in U.S. Pat. No. 7,082,336. In one approach, the lead includes a cuff, through which a guidewire is introduced through a distal end of the lead while the lead is outside of the body and the device is already implanted. The guidewire is steered to the fixation site, and a pusher is introduced onto the free end of the wire. The pusher is advanced against the lead cuff, and the lead is pushed along the guidewire to the fixation location. A fixation element is provided on the lead tip for securing the lead at its desired location.

While intravascular implantable devices represent a significant improvement over conventional long-term active implantable devices that are implanted subcutaneously, there are opportunities to improve and refine the implantation techniques, system and tools for implanting the cardiac leads associated with such intravascular devices. It would be desirable to provide improved methods, systems and tools for implanting such intravascular implantable devices that can simplify the implantation of these devices so as improve the effectiveness and ease of the procedure.

### SUMMARY OF THE INVENTION

The present invention is directed to methods, systems and tools for implanting leads associated with long-term active therapeutic medical devices referred to as an intravascular implantable device (IID) within a patient's vasculature. Implantation of an IID generally includes maintaining a vessel puncture open during the procedure, delivery and placement of the device, delivery and fixation of one or more anchors to retain the device within the vasculature, and delivery and fixation of one or more leads, with these procedures not necessarily carried out in this order.

The IID may be provided with a single lead integrally connected into the proximal end of the device body. Delivery and implantation of the lead is typically performed subsequent to the device delivery. To deliver a lead from the inferior vena cava to the interior of the heart, such as into the right atrium or right ventricle, the lead must be maneuvered through the acute angle between the inferior vena cava and the heart. Suitable lead delivery systems provide positive control of the lead, or at least the distal end of the lead, during implantation.

In one embodiment, the lead delivery system comprises a grasper-style device configured to releasably grasp the distal end of the lead. The grasper device includes the ability to articulate, rotate and/or extend to facilitate delivery of the lead to the desired location. In one embodiment, the grasper-style lead delivery system includes an elongated flexible body adapted to be temporarily implanted into a patient's vasculature. The elongate flexible body includes a proximal end and a distal end and can include a flexible wire. A handle is operably connected to the proximal end of the device body and a grasper mechanism can be connected to the distal end of the flexible wire. The grasper mechanism can be configured to releasably grasp a cardiac lead by closing a releasable honda that is selectively controllable with the handle around the lead. Articulation of the distal end of the lead delivery system can be accomplished through the use of one or more pull wires extending internally from the distal end of the device to the handle. The lead delivery system may be configured such that rotating the handle with respect to the flexible body pulls the internal wire, causing the articulation of the distal end of lead delivery system. In another embodiment, a supplemental thumb slide may be provided in handle, operably coupled to pull wire to cause articulation of the distal end.

In one embodiment, the releasable honda can be securely closed around the lead by inserting an end portion of the releasable honda into a collar section located at the distal end of the lead delivery system body. In another embodiment, the releasable honda can be closed around the lead by connecting a stylet projecting from device body with the releasable honda. The releasable honda can be configured to release its grasp on the lead via operation of the handle, or in another embodiment, through the use of a stylet.

In another embodiment, the invention comprises a method of implanting a lead within a heart, wherein the proximal end of the lead does not provide access to an internal lumen suitable for stylet introduction. A lead delivery system having a releasable lasso is used to grasp the lead, preferably at or near the distal end of the lead. The lead is advanced through the vasculature to the desired implant location with the lead delivery system, such as by articulating, rotating, and/or extending the lead delivery system. In one embodiment, the lead is implanted within the heart from a location within the inferior vena cava. To facilitate implantation from the inferior vena cava, the distal end of the lead is grasped with a releasable lasso portion of a lead delivery system, and advanced toward the heart. Upon reaching the heart, lead delivery system is manipulated to navigate the distal end of the lead through the acute angle formed by the inferior vena cava and the right atrium. The use of articulation, rotation, extension, or any combination thereof, may be required to guide the lead into the heart and onto a desired implant location within the heart.

In another embodiment of implanting a lead within a heart, lead delivery system body is provided with an internal lumen for receipt of a guidewire or stylet. A pre-shaped guidewire may be delivered with a separate guidewire catheter, the pre-shaped guidewire creating a path from the desired lead implant location within the heart, through the inferior vena cava, and out the introducer sheath. After the guidewire catheter has been withdrawn, the lead is grasped by lead delivery system and advanced over the guidewire to the desired implant location.

The above summary of the various embodiments of the invention is not intended to describe each illustrated embodiment or every implementation of the invention. This summary represents a simplified overview of certain aspects of the invention to facilitate a basic understanding of the invention and is not intended to identify key or critical elements of the invention or delineate the scope of the invention.

### BRIEF DESCRIPTION OF THE DRAWINGS

The invention may be more completely understood in consideration of the following detailed description of various embodiments of the invention in connection with the accompanying drawings, in which:

FIG. 1 is a perspective illustration depicting human cardiac anatomy.

FIG. 2 is a schematic generally depicting components of an intravascular electrophysiological system according to one aspect of the present invention.

FIG. 3 is a schematic of one embodiment of an intravascular implantable device according to the present invention.

FIG. 4A is a perspective view of a lead delivery system according to one aspect of the present invention.

FIG. 4B is an exploded perspective view of the lead delivery system of FIG. 4A.

FIG. 4C is a top plan view of the lead delivery system of FIG. 4A.

FIG. 4D is a side plan view of the lead delivery system of FIG. 4A.

FIG. 4E is an end plan view of the lead delivery system of FIG. 4A.

FIG. 5A is a perspective view of a device body of a lead delivery system according to one aspect of the present invention.

FIG. 5B is a top plan view of the device body of FIG. 5A, depicted without a sheath for clarity.

FIG. 5C is a side plan view of the device body of FIG. 5B.

FIG. 5D is a top plan view of the device body of FIG. 5A.

FIG. 5E is a side plan view of the device body of FIG. 5A.

FIG. 6A is a view of an intravascular implantable electrophysiology device being guided through the inferior vena cava of a patient according to one aspect of the present invention.

FIG. 6B is a view of the intravascular implantable electrophysiology device of FIG. 6A positioned fully within the vasculature of the patient, with a cardiac lead extending from the proximal end of the device and being grasped by a lead delivery system according to one aspect of the present invention.

FIG. 6C is a view of a cardiac lead being guided through the vasculature with a lead delivery system according to one aspect of the present invention.

FIG. 6D is a view of a cardiac lead being guided to a desired location within the heart of a patient by a lead delivery system according to one aspect of the present invention.

FIG. 7A is a perspective view of a grasper mechanism according to one aspect of the present invention in a free state.

FIG. 7B is a perspective view of the grasper mechanism of FIG. 7A in an engaged state.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE DRAWINGS

In the following detailed description of the present invention, numerous specific details are set forth in order to provide a thorough understanding of the present invention. However, one skilled in the art will recognize that the present invention may be practiced without these specific details. In other instances, well-known methods, procedures, and components have not been described in detail so as to not unnecessarily obscure aspects of the present invention.

The present disclosure describes intravascular electrophysiological systems that may be used for a variety of functions. These functions include defibrillation, pacing and/or cardioversion. In general, the elements of the systems described herein include at least one device body and typically, but optionally, at least one lead coupled to the body. One or more retention devices may facilitate the retension of the device body and/or leads or other elements within the vasculature. Also described are components such as mandrels, stylets and/or guide wires used to facilitate implantation of the system.

### Anatomy

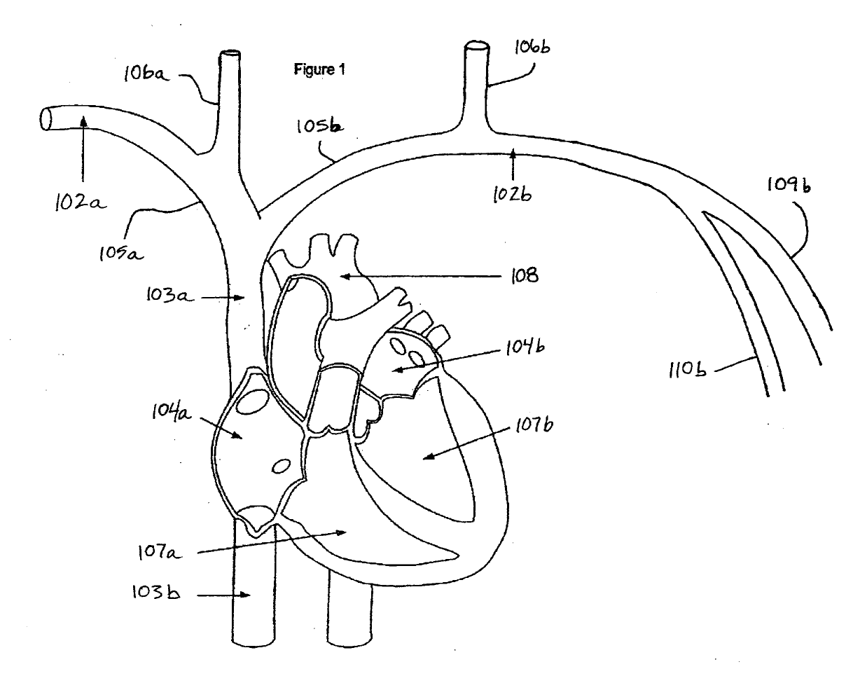

Referring to FIG. 1, the general cardiac anatomy of a human is depicted, including the heart and major vessels. The following anatomic locations are shown and identified by the listed reference numerals: Right Subclavian 102a, Left Subclavian 102b, Superior Vena Cava (SVC) 103a, Inferior Vena Cava (IVC) 103b, Right Atrium (RA) 104a, Left Atrium (LA) 104b, Right Innominate/Brachiocephalic Vein 105a, Left Innominate/Brachiocephalic Vein 105b, Right Internal Jugular Vein 106a, Left Internal Jugular Vein 106b, Right Ventricle (RV) 107a, Left Ventricle (LV) 107b, Aortic Arch 108, Descending Aorta 109, Right Cephalic Vein 109a (not shown in FIG. 1), Left Cephalic Vein 109b, Right Axillary Vein 110a (not shown in FIG. 1) and Left Axillary Vein 110b. Reference number 100 refers generally to vessels and/or vessel walls within the human body.

### The Kit

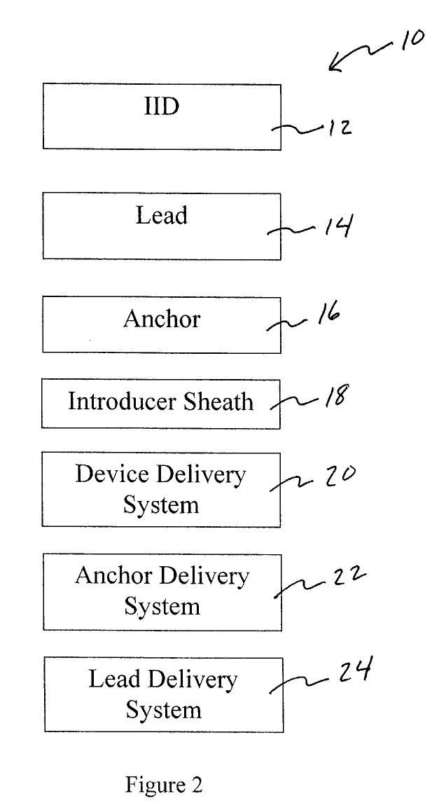

One configuration of the components of an electrophysiological treatment system 10 is depicted in FIG. 2. System 10 generally includes an intravascular implantable device (IID) 12 having a lead 14, the device being retained within a vessel 100 by an anchor 16. An introducer sheath 18 is provided for implantation of system 10. A guidewire catheter 20 is provided to deploy a guidewire 22 within the vasculature of a patient. A device delivery system 24 may be used in conjunction with guidewire 22 to navigate the IID to the desired location. An anchor delivery system 26 may also rely on guidewire 22 to deliver anchor 16 to the desired location. Anchor delivery system 26 may also include a means for fixing or deploying anchor 16. A lead delivery system 28 is provided for maneuvering lead 14 to its desired location.

In one embodiment, instructions for implanting the system 10 in accordance with the various embodiments described herein in the form of printed or electronically, optically or magnetically stored information to be displayed, for example, are provided as part of a kit or assemblage of items prior to surgical implantation of the system 10. In another embodiment, instructions for implanting the system 10 in accordance with the various embodiments described herein are provided, for example, by a manufacturer or supplier of system 10, separately from providing the system 10, such as by way of information that is accessible using the Internet or by way of seminars, lectures, training sessions or the like.

### Structure of the Intravascular Implantable Device

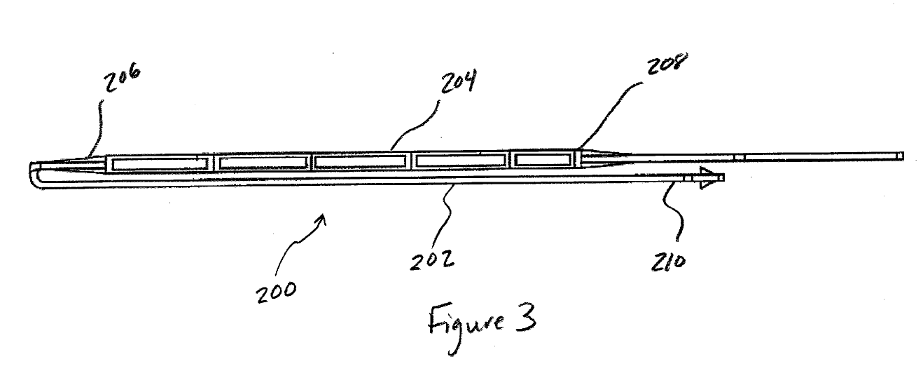

Referring to FIG. 3, an intravascular implantable device (IID) 200 according to one aspect of the present invention is depicted. In one embodiment, IID 200 includes components known in the art to be necessary to carry out the system functions. For example, IID 200 may include one or more pulse generators, including associated batteries, capacitors, microprocessors, and circuitry for generating electrophysiological pulses for defibrillation, cardioversion and/or pacing. IID 200 also includes detection circuitry for detecting arrhythmias or other abnormal activity of the heart. The specific components to be provided in the device will depend upon the application for the device, and specifically whether the device is intended to perform defibrillation, cardioversion and/or pacing along with its sensing functions.

IID 200 is proportioned to be passed into the vasculature and to be anchored within the patient's vasculature with minimal obstruction to blood flow. Suitable sites for IID 200 may include, but are not limited to, the venous system using access through the right or left femoral vein or the subclavian or brachiocephalic veins, or the arterial system using access through one of the femoral arteries. Thus, the housing of IID 200 preferably has a streamlined maximum cross sectional diameter which may be in the range of 3-15 mm or less, with a most preferred maximum cross-sectional diameter of 3-8 mm or less. The cross-sectional area of the device 200 in the transverse direction (i.e. transecting the longitudinal axis) should be as small as possible while still accommodating the required components. The cross-section of device 200 (transecting the longitudinal axis) may have a circular cross-section, although other cross-sections including crescent, flattened, or elliptical cross-sections may also be used. It can be desirable to provide the device with a smooth continuous contour so as to avoid voids or recesses that could encourage thrombus formation on the device.

Cardiac lead 202 may be integrated with the device body 204, or lead 202 may be integral with device 200 as an extension of the device itself. Multiple leads 202 may be provided. Leads 202 may be included on the proximal end 206 of device body 204, on the distal end 208 of device body 204, generally on device body 204, and/or any combination thereof. Lead 202 includes one or more defibrillation and/or pacing electrodes 209 and may also be equipped to sense electrical activity of the heart. Monitoring of the heart's electrical activity can be needed to detect the onset of an arrhythmia. Activity sensed by the sensing electrode(s) is used by the device electronics to trigger delivery of a defibrillation shock. The lead 202 may be a conventional defibrillation/pacing lead, although alternative lead configurations may be desirable if warranted by the desired placement of the IID 200 and lead 202 within the body.

For leads 202 that are to be positioned within a chamber of the heart, the leads 202 may be the helical screw-in or tined variety for fixation to the cardiac tissue, and/or they may have steroid-eluding tips to facilitate tissue in-growth for fixation purposes. If a detachable tip is used, lead tip 210 may be left within the chamber of the heart when the remainder of lead 202 is removed, so as to prevent damage to the heart tissue as could occur upon extraction of the tined tip.

The leads 202 may include non-thrombogenic and/or non-proliferative surfaces or coatings, for example, the leads 202 may include a coating that is anti-thrombogenic (e.g. perfluorocarbon coatings applied using supercritical carbon dioxide) so as to prevent thrombus formation on the lead 202. It is also beneficial for the coating to have anti-proliferative properties so as to minimize endothelialization or cellular ingrowth, since minimizing growth into or onto the lead 202 will help minimize vascular trauma when the device is explanted. The coating may thus also be one which elutes anti-thrombogenic compositions (e.g. heparin sulfate) and/or compositions that inhibit cellular in-growth and/or immunosuppressive agents.

Thus, it should be appreciated that in this disclosure the term “lead” is used to mean an element that includes conductors and electrodes and that thus may be positioned somewhat remotely from the circuitry that energizes the electrodes. In other embodiments, leads may include elements that are simply extensions or tapers of the IID itself (such as the portion of the device at which electrodes are located) as well as more conventional intravascular leads.

In accordance with one embodiment of the present invention, IID 200 can include at distal end 208 an anchor attachment feature that allows IID 200 to be disposed within the vasculature. An anchor detachment feature may be included so as to allow for removal of IID 200 at a later date without damaging the vasculature by removing the anchor. An anchor zone may be disposed between the anchor attachment feature and the detachment feature for positioning IID 200 between an anchor and the vasculature wall. In one embodiment, a telemetry antenna may be disposed axially along distal end 208 proximate the anchor zone.

IID 200 may also be provided with a lumen for passage of a guidewire therethrough, such as described in U.S. Published Application Nos. 2008/0147168 and 2008/0167702, the disclosures of which are hereby incorporated by reference. The lumen may be included in distal portion 208 of the device body 204. In another embodiment, IID 200 includes a tip portion coupled to distal portion 208 of device body 204. Tip portion may include an internal telemetry antenna, a guidewire lumen extending the length of the tip, and tip portion may further provide an anchor attachment feature.

Additional disclosure pertaining to the structure and layout of intravascular implantable devices, as well as leads and anchors, can be found in U.S. Published Patent Application Nos. 2006/0217779, 2007/0265673, 2008/0147168, and 2008/0167702, the disclosures of which are hereby incorporated by reference.

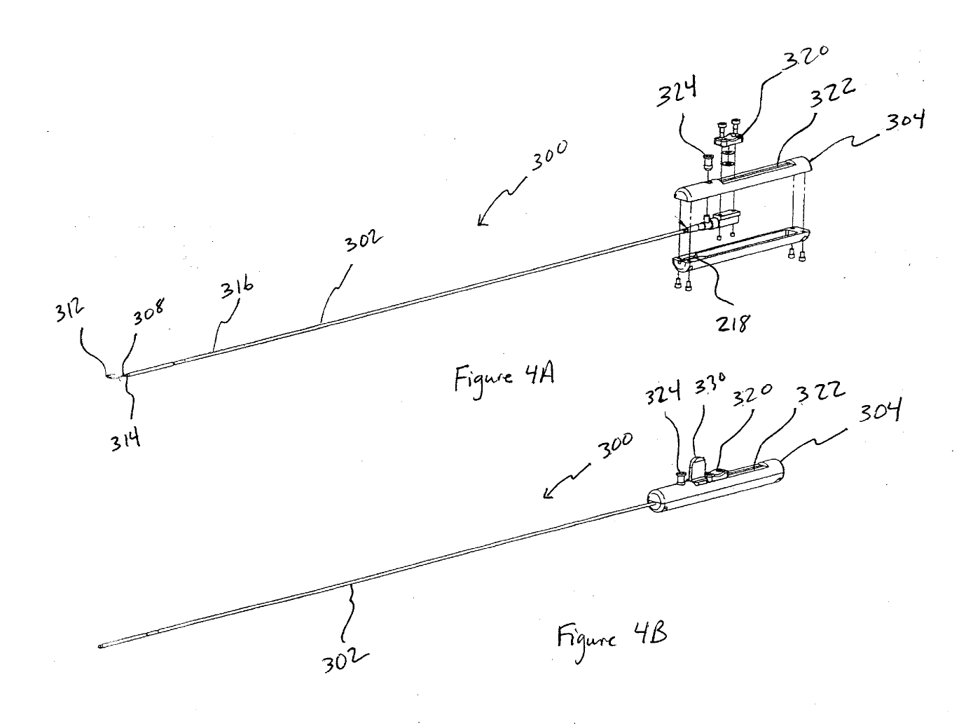

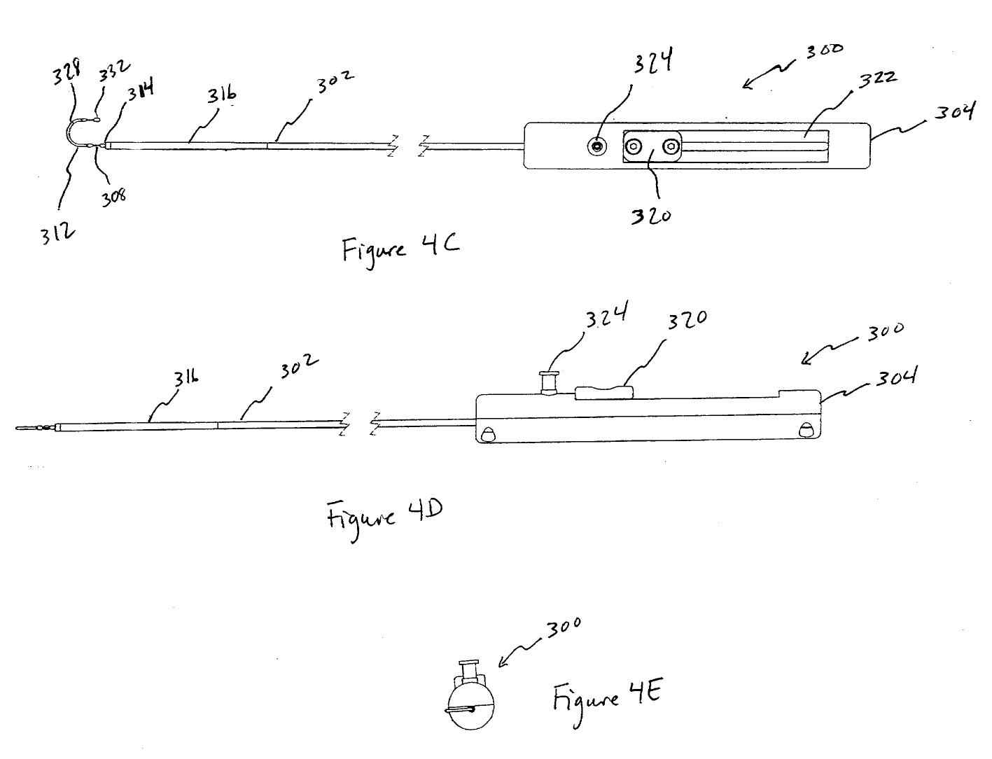

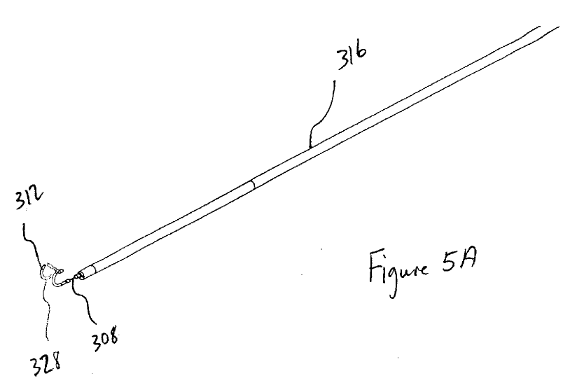

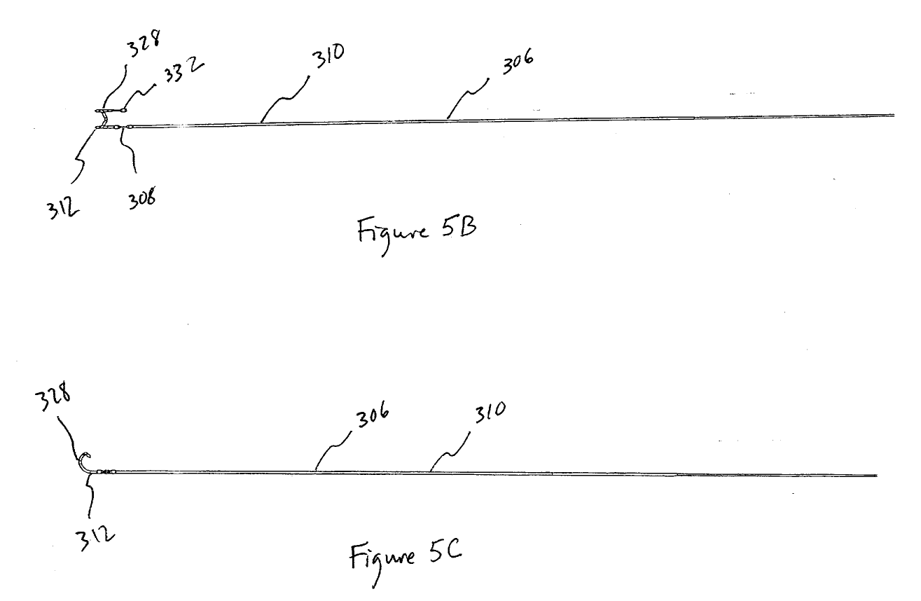

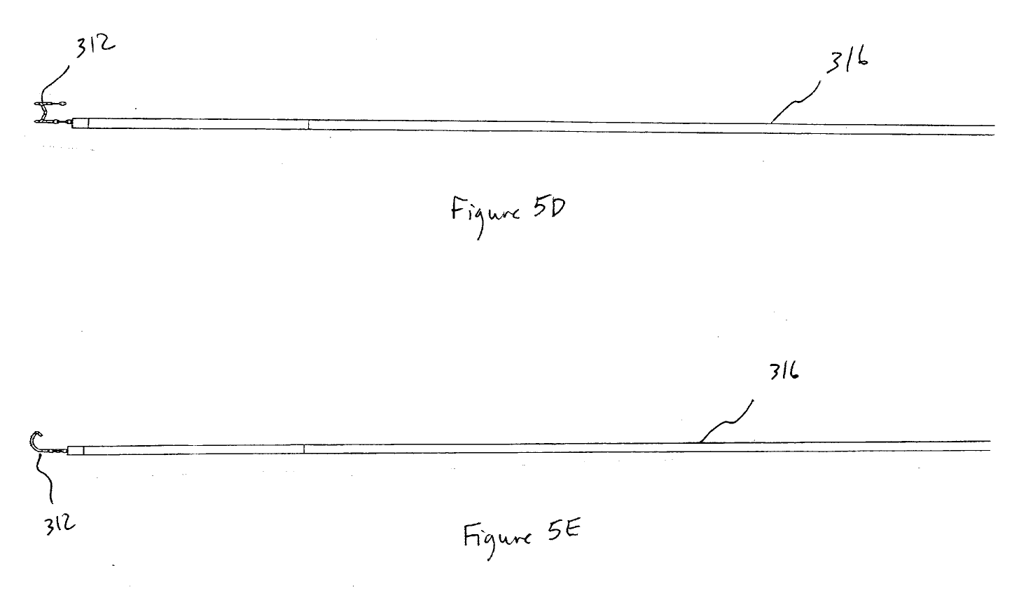

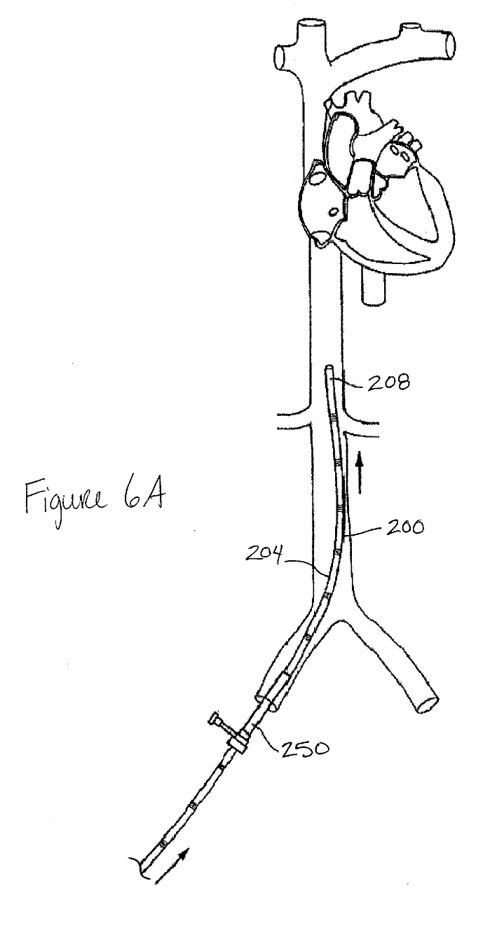

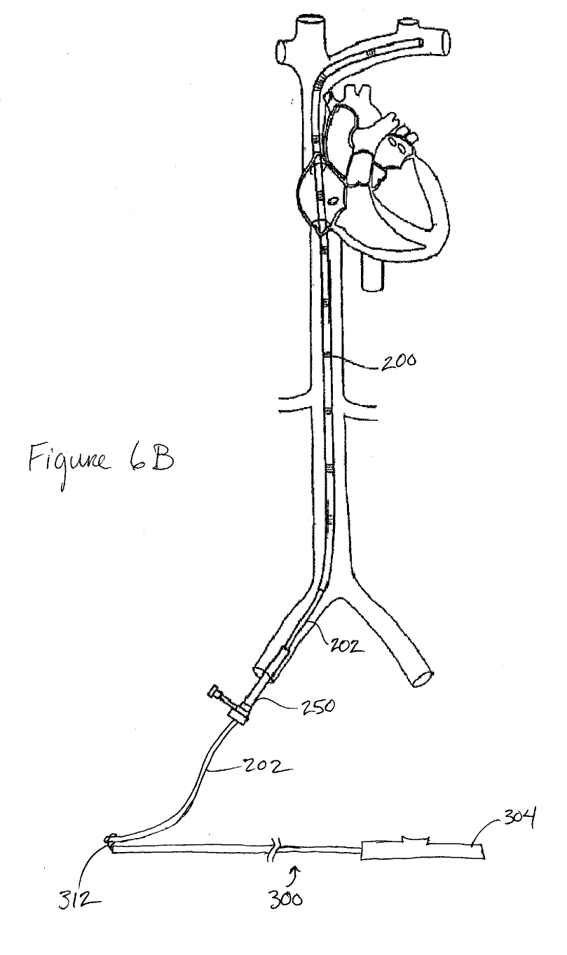

Embodiments of a lead delivery system 300 are depicted in FIGS. 4A-4E and 5A-5E. Lead delivery system 300 can generally include a device body 302 and a handle 304.

Device body 302 includes a flexible wire 306. Wire 306 can be a coaxial wire that includes an inner wire 308, and an outer wire 310 that generally surrounds inner wire 308. A grasper mechanism 312 may be disposed at a distal end of the inner wire 308. Device body 302 can also include a flexible sheath 316 surrounding wire 306. The distal end of the flexible sheath 316 can include a collar section 314. The collar section 314 can have an inner diameter sized to incorporate not only the wire 306, but also the grasper mechanism 312. A secondary wire, or stylet, can also be contained in device body. Stylet can be contained within flexible sheath 316 in a separate lumen from wire 306, or can be contained in the same aperture as wire 306.

Handle 304 can define a central longitudinal aperture 318 that can accommodate body 302. A thumb slide 320 can be disposed within an axially positioned slot 322. Thumb slide 320 can be connected to a proximal end of wire 306. Handle 304 can also include a flush port 324, which is adapted to flush blood out of delivery system 300 during implantation.

The grasper mechanism 312 has a lasso-like, or collar shape that permits selective frictional or pressure based grasping and releasing of the IID 200 without the need for a positive mechanical mating of a mandrel as with prior device delivery solutions. The lasso (or lariat) 327 includes a releasable loop 328, or honda, portion. The releasable honda 328 is configured to grasp the IID in order to position the IID 200 in the vasculature. Releasable honda 328 has an end portion 332 that can be connected to the collar section 314 to form a closed loop. The grasper mechanism 312 can be releasably coupled to the proximal end of the IID 200, the distal end of the IID 200, the lead 202, or any other portion of the IID 200 in order to position the IID 200 in the vasculature. In one embodiment, the IID 200 includes a circumferential notch around the device body 204 configured to be grasped by the lead delivery system 300. The grasper mechanism 312 can have various shapes. The embodiment shown in FIGS. 4A-4E is generally “u” or hook shaped. Another embodiment of a grasper mechanism 312 suitable for device delivery depicted in FIGS. 5A-5E, is generally “w” shaped. Any shape that can be used with the collar section or a stylet to form a closed loop to grasp an IID can be used.

In one embodiment, grasper mechanism 312 can be formed of memory wire, such as nitinol. The shape of the grasper mechanism 312 can be formed by setting the memory wire in a heated fixture. Memory wire allows the grasper mechanism to deform when necessary while still naturally retaining the grasping shape.

The thumb slide 320 can move within the axially disposed slot 322 in the handle 304. If the proximal end of inner wire 308 is connected to thumb slide 320, axial movement of the thumb slide 320 produces a corresponding movement of the inner wire 308. A stop fixture 330 can be disposed at the distal end of the slot 322, to prevent the thumb slide 320 from moving past a certain point. The stop fixture 330 reduces the potential for the inadvertent release of the IID 200 during the implantation procedure. Alternatively, the thumb slide 320 can control the operation of a stylet contained within sheath 316 that is separate from wire 306. Alternatively, thumb slide 320 can be switched to operate an extension assembly coupled to outer wire 310, the extension assembly adapted to provide extension of the grasper mechanism.

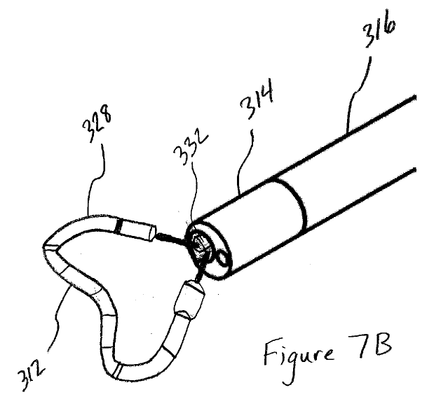

The grasper mechanism 312 can be configured to grasp and release the IID 200 in various ways. In one embodiment, as can be seen in FIGS. 7A and 7B, the end portion 332 of releasable honda 328 of grasper mechanism 312 is manually inserted into collar section 314 of device body 302 to form a closed loop for grasping the IID 200. This can be done outside of the body on the operating table. To release the IID 200, the stop fixture 330 is removed allowing thumb slide 320 to advance into the region previously occupied by the stop fixture 330, pushing the inner wire 308 forward. As the inner wire 308 slides as far forward as it can go, the end portion 332 of releasable honda 328 is pulled out of collar section 314, thereby releasing the IID 200 from the grasp of lead delivery system 300.

In another embodiment, a closed loop is formed around an IID 200 by mating a stylet with the end portion 332 of releasable honda 328. To open the loop, the stop fixture 330 can be removed and the thumb slide 320 can move forward to push the inner wire 308 and end portion 332 out of contact with the stylet. Alternatively, the thumb slide 320 can control movement of the stylet and the loop can be opened by withdrawing the stylet out of contact with the end portion 332. In this alternative, it is possible to remotely grasp the IID 200 while it is in or out of the body, by remotely moving the stylet forward with thumb slide 320 to close the loop with the end portion 332 around IID 200.

### Implantation Methods

Implantation of an IID 200 generally includes maintaining a vessel puncture open during the procedure, delivery and placement of the device body 204, delivery and fixation of one or more anchors to retain the device within the vasculature, and delivery and fixation of one or more leads 202, with these procedures not necessarily carried out in this order.

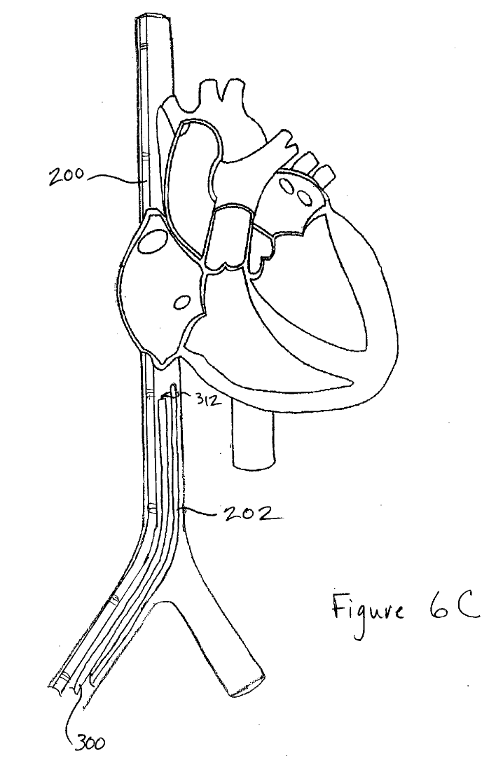

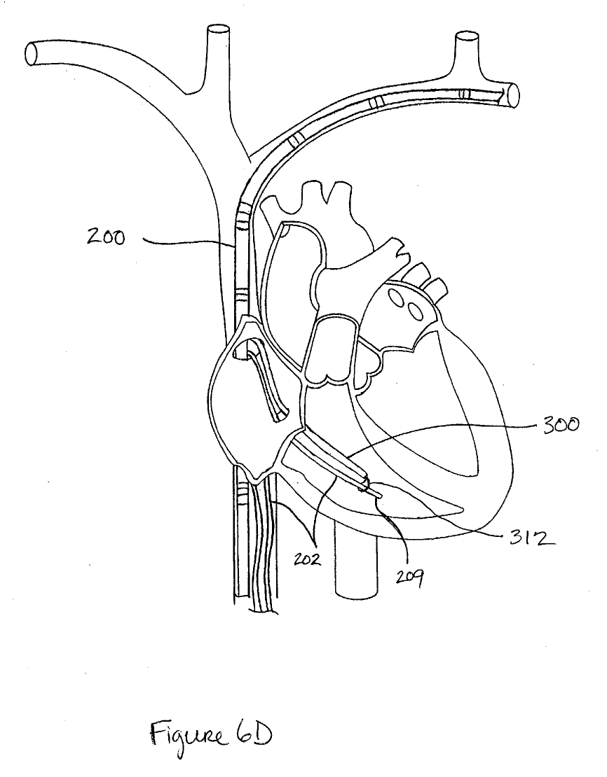

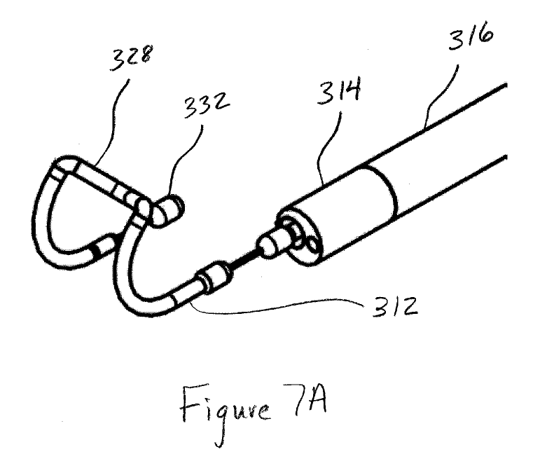

Referring now to FIGS. 6A-6D, one embodiment of lead implantation is depicted wherein implantation of lead 202 occurs subsequent to the delivery and placement of the IID 200 to which lead 202 is attached. IID 200 is positioned such that the proximal end of lead 202, being integrally formed with the proximal end 206 of IID 200, is located generally within or near the inferior vena cava. Utilizing the grasper-style lead delivery system 300, the distal end of lead 202 is securely grasped by releasable honda 328. Distal end of lead 202 may be situated within the vasculature, or may be extending through the introducer sheath and residing at least partly outside of the patient, as depicted in FIG. 6B. Lead 202 may be grasped at or near its distal end, to provide positive directional control of the distal end of the lead during implantation. The precise grasping location on distal end of lead 202 will be dependent on the particular structure of lead 202, such as the location of any electrodes or fixation elements.

Once lead 202 is securely grasped, lead delivery system 300 is used to navigate lead 202, distal end first, through the inferior vena cava toward the heart, as depicted in FIG. 6C. To guide lead 202 into the heart from the inferior vena cava, lead delivery system 300 may be rotated, articulated, extended or any combination thereof so as to navigate the acute angle from the inferior vena cava into the right atrium and avoid damage or interference with tissue in and around the heart. Additional manipulation of lead delivery system 300 may be required to guide lead 202 to its desired location within the heart, as depicted in FIG. 6D.

In another embodiment of lead implantation, lead 202 may be partially delivered to its desired location with lead delivery system 300, released from delivery system 300, and re-grasped at another portion of lead 202. Such method permits grasping lead 202 at a desired position during a first phase of implantation, and at a second position during a second phase of implantation.

In a further embodiment, lead delivery system 300 is provided with an internal lumen configured for receipt of a guidewire or steerable stylet. A pre-shaped guidewire may be first implanted with a guidewire introducer catheter, the pre-shaped guidewire defining a pathway from the desired implant location for lead 202 within the heart, through the inferior vena cava, and out the introducer sheath positioned in the femoral vein. After withdrawing the guidewire introducer catheter, lead 202 is grasped with releasable honda 328 and lead delivery system 300 is advanced onto the guidewire. Lead delivery system 300 with lead 202 releasably attached is introduced along the guidewire to the desired implant location. During implantation, if needed the guidewire can be removed from the lumen in lead delivery system 300, reshaped, and then reinserted into the lumen in lead delivery system 300 so as to alter the path along which lead delivery system 300 and lead 202 are advanced.

In another embodiment wherein lead delivery system 300 includes an internal lumen, a steerable stylet is provided for assisting in delivery of lead 202. Releasable honda 328 is used to grasp lead 202, and lead delivery system 300 and lead 202 are positioned within the inferior vena cava. The steerable stylet is introduced in the proximal end of lead delivery system body 204. As lead delivery system 300, with lead 202 attached, is advanced through the vasculature, steerable stylet may be used to control, or supplement the control of, the direction of lead delivery system 300.

Upon successful delivery of lead 202 to its desired location, in one embodiment lead 202 is released from lead delivery system 300 to allow for passive fixation of lead 202. In another embodiment, lead delivery system 300 is withdrawn prior to fixation of lead 202, so as to allow introduction of a fixation device for securing the lead in place. Withdrawal of lead delivery system 300 includes releasing honda 328 from lead 202, such as by releasing stop fixture 330 and operating thumb slide 320 to open the lasso. Thumb slide can further be operated to completely withdraw grasper mechanism 312 into the device delivery system 300 device body 302, to prevent grasper mechanism 312 from contacting vessel walls during withdrawal.

In another embodiment wherein lead delivery system 300 includes a lumen for a stylet, a stylet is introduced through the lumen to the lasso, and used to disengage end portion 332 of releasable honda 328 from collar section 314 of device body 302.

Various embodiments of systems, devices and methods have been described herein. These embodiments are given only by way of example and are not intended to limit the scope of the present invention. It should be appreciated, moreover, that the various features of the embodiments that have been described may be combined in various ways to produce numerous additional embodiments. Moreover, while various materials, dimensions, shapes, implantation locations, etc. have been described for use with disclosed embodiments, others besides those disclosed may be utilized without exceeding the scope of the invention.

Persons of ordinary skill in the relevant arts will recognize that the invention may comprise fewer features than illustrated in any individual embodiment described above. The embodiments described herein are not meant to be an exhaustive presentation of the ways in which the various features of the invention may be combined. Accordingly, the embodiments are not mutually exclusive combinations of features; rather, the invention may comprise a combination of different individual features selected from different individual embodiments, as understood by persons of ordinary skill in the art.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims for the present invention, it is expressly intended that the provisions of Section 112, sixth paragraph of 35 U.S.C. are not to be invoked unless the specific terms “means for” or “step for” are recited in a claim.

1. A method of implanting an intravascular implantable electrophysiological device having a device body and a cardiac lead coupled to the device body that are adapted for implantation within the vasculature of a patient, comprising:
implanting the device body within the vasculature of a patient, the device body including circuitry adapted to deliver electrophysiological therapy through the cardiac lead, the cardiac lead having a distal end including an electrode and being adapted for fixation within a heart of the patient, and a proximal end non-releasably coupled to a proximal end of the device body;
with at least a portion of the lead external to the patient, releasably grasping an exterior of the lead proximate the distal end of the lead with a lead delivery system; and
utilizing the lead delivery system to implant the cardiac lead by:
delivering the distal end of the cardiac lead through the vasculature to a desired location within the heart of the patient;
fixating the distal end of the cardiac lead at the desired location; and releasing the cardiac lead from the lead delivery system and removing the lead delivery system from the patient.

2. The method of claim 1, wherein delivering the distal end of the cardiac lead comprises delivering the cardiac lead through the inferior cava and into the right ventricle.

3. The method of claim 2, wherein delivering the cardiac lead through the inferior cava and into the right ventricle further comprises delivering the cardiac lead proximate the right atrium, releasing the cardiac lead from the lead delivery system, repositioning the lead delivery system, grasping the cardiac lead with the lead delivery system, and delivering the cardiac lead through the right atrium into the right ventricle.

4. A method of implanting an intravascular implantable electrophysiological device, comprising:
providing an intravascular implantable electrophysiological device having a device body and a cardiac lead coupled to the device body that are adapted for implantation within the vasculature of a patient, the device body including circuitry adapted to deliver electrophysiological therapy through the cardiac lead, the cardiac lead having a distal end including an electrode and being adapted for fixation within a heart of the patient and a proximal end non-releasably coupled to a proximal end of the device body;
providing a lead delivery system having an elongated flexible body, a handle operably coupled to a proximal end of the flexible body, and a grasper mechanism operably coupled to a distal end of the flexible device body; and
providing instructions, including:
implanting the device body within the vasculature of a patient;
with at least a portion of the lead external to the patient, releasably grasping an exterior of the lead proximate the distal end of the lead with a lead delivery system; and
delivering the distal end of the cardiac lead through the vasculature with the lead delivery system to a desired location within the heart of the patient;
fixating the distal end of the cardiac lead at the desired location; and
releasing the cardiac lead from the lead delivery system and removing the lead delivery system from the patient.

5. A method of implanting a cardiac lead associated with an intravascular implantable electrophysiological device, wherein a proximal end of the cardiac lead is non-releasably coupled to the proximal end of the device and the proximal end of the device is situated in an inferior vena cava of a patient, comprising:
releasably grasping an exterior of the cardiac lead with a lead delivery system;
delivering the lead through a vasculature of the patient with the lead delivery system to a desired location within the heart;
fixating the lead at a desired location within the heart of the patient; and
releasing the cardiac lead from the lead delivery system and removing the lead delivery system.

6. The method of 5, wherein delivering the lead further comprises manipulating the lead delivery system so as to direct the cardiac lead from the inferior vena cava into a right atrium of the heart of the patient;

7. A method of implanting a cardiac lead associated with an intravascular implantable electrophysiological device, comprising:
providing an intravascular implantable electrophysiological device having a device body and a cardiac lead, wherein a proximal end of the cardiac lead is non-releasably coupled to a proximal end of the device body and wherein the proximal end of the device body is situated in an inferior vena cava of the patient;
providing a lead delivery system having an elongated flexible body, a handle operably coupled to a proximal end of the flexible body, and a grasper mechanism operably coupled to a distal end of the flexible device body; and
providing instructions, including:
releasably grasping an exterior of the cardiac lead with the grasper mechanism of the lead delivery system;
delivering the cardiac lead through a vasculature of the patient with the lead delivery system to a desired location within the heart;
fixating the lead at a desired location within the heart of the patient; and
releasing the cardiac lead from the lead delivery system and removing the lead delivery system.
